(19) United States
(12) Patent Application Publication
(10) Pub. No.: US 2023/0167401 A1
(43) Pub. Date: Jun. 1, 2023

(54) BACTERIUM OF THE CHRISTENSENELLACEAE FAMILY FOR THE PREVENTION AND/OR TREATMENT OF CHRONIC INFLAMMATORY DISEASES AND/OR INFLAMMATORY GASTROINTESTINAL DISEASES AND/OR CANCERS

(71) Applicants: YSOPIA BIOSCIENCES, BORDEAUX CEDEX (FR); INSTITUT NATIONAL DE RECHERCHE POUR L'AGRICULTURE, L'ALIMENTATION ET L'ENVIRONNEMENT, PARIS (FR); ALMA MATER STUDIORUM UNIVERSITÀ DI BOLOGNA, BOLOGNA (FR)

(72) Inventors: Georges RAWADI, PARIS (FR); Sandrine Paule CLAUS, BORDEAUX (FR); Laure RINALDI, BORDEAUX (FR); Frédéric ELUSTONDO, BORDEAUX (FR); Marion Sophie, Madeleine SOTO, MERIGNAC CEDEX (FR); Katy Nicole LECORF, MERIGNAC (FR); Camille Muriel, Dominique, Mathilde KROPP, MEUDON (FR); Philippe LANGELLA, VELIZY-VILLACOUBLAY (FR); Rebeca MARTIN ROSIQUE, JOUY EN JOSAS (FR); Patrizia BRIGIDI, BOLOGNA (FR)

(21) Appl. No.: 17/437,492

(22) PCT Filed: Mar. 11, 2020
(86) PCT No.: PCT/EP2020/056565

§ 371 Date: Sep. 9, 2021

Related U.S. Application Data
(60) Provisional application No. 62/816,539, filed on Mar. 11, 2019.

(30) Foreign Application Priority Data
Mar. 11, 2019 (FR) 1902437
Jul. 24, 2019 (FR) 1908384

(51) Int. Cl. C12N 1/20; A61K 35/74; A61P 29/00; A61P 1/00; A61K 35/741; A61K 45/06

(52) U.S. Cl. CPC C12N 1/20; A61K 35/74; A61P 29/00; A61P 1/00; A61K 35/741; A61K 45/06; C12N 2500/74; C12N 2500/80; C12N 2500/02; C12N 2500/34; C12N 2500/32; A61K 2035/115

(57) ABSTRACT

The invention relates to a bacterium of the Christensenellaceae family, in particular of the genus Christensenella, or a composition containing same for use in the prevention and/or treatment of chronic inflammatory diseases and/or cancers in humans or animals.

### CROSS-REFERENCE TO RELATED APPLICATIONS

This application is a U.S. National Stage Application of PCT/EP2020/056565 assigned the international filing date of Mar. 11, 2020 and claiming the benefit of priority from U.S. Provisional patent application 62/816,539 filed Mar. 11, 2019 and also claiming a foreign priority from EP patent application 1902437 filed Mar. 11, 2019 and from EP patent application 1908384 filed Jul. 24, 2019, the disclosure of all these applications is herein incorporated by reference.

### INCORPORATION BY REFERENCE OF THE SEQUENCE LISTING

The content of the ASCII text file of the sequence listing named 1A00020.txt, 10.1 kb in size, created on Feb. 15, 2022, is incorporated herein by reference in its entirety.

### TECHNICAL FIELD

The invention relates to the prevention and treatment of chronic inflammatory diseases, inflammatory gastrointestinal diseases and/or cancers with specific bacteria of the intestinal microbiota and compositions thereof.

### BACKGROUND

Inflammation is the body's normal defense process against aggression. It makes it possible to fight and eliminate the foreign agent(s) at the origin of the said aggression. This inflammation, known as acute, is reversible and has only a limited duration ranging from a few minutes to a few days. However, in some cases the inflammatory process goes wrong and the inflammation becomes chronic. Instead of contributing to the body's defense, the actors involved in the inflammatory process become dangerous and lead to an often serious and disabling pathological condition. Chronic inflammatory diseases are therefore diseases whose pathophysiology is directly linked to inflammation, said inflammation being of autoimmune and/or auto-inflammatory origin.

All organs can be affected by chronic inflammation, such as the digestive system (Crohn's disease, ulcerative colitis, autoimmune gastritis, etc.), the liver (hepatitis, NAFLD, non-alcoholic steatohepatitis, etc.), pancreas (pancreatitis), lungs (asthma), skin (atopic dermatitis such as psoriasis), nervous system (multiple sclerosis), joints (polyarthritis). Moreover, chronic inflammation is also present in other situations where other mechanisms are involved, such as cancer in particular. Chronic inflammation, and in particular chronic inflammatory diseases, affect a large portion of the population, in varying proportions depending on the pathology concerned. The consequences for health are important, because these diseases, although slowly evolving, are disabling, painful and associated with a high risk of early mortality.

Currently, the treatments proposed for these diseases are anti-inflammatory drugs, immunomodulators and/or immunosuppressants. We can cite for example:
non-steroidal anti-inflammatory drugs such as ibuprofen, naproxen and aspirin which are intended for short-term use and which are unsuitable for chronic pain,
purine metabolism modulators which are immunomodulators, such as methotrexate and azathioprine,
biological drugs such as anti-TNF-α, antagonists of the IL1 and IL6 receptors, or even
sulfasalazine.

All of these drugs have very variable response rates due to the degradation of the molecules when they are administered and have significant side effects. In addition, immunomodulators interfere with the normal and healthy immune response to pathogens, so that treated patients are susceptible to infections. As for methotrexate, it is specifically known for its adverse effects on sperm production and therefore on fertility. Thus, most of these inflammatory diseases go untreated or without adequate treatment.

Therefore, the study and research of new anti-inflammatory treatment strategies constitute a major focus in medicine and biomedical research, in particular the development of an effective treatment of chronic inflammatory diseases, gastrointestinal diseases and diseases induced by chronic inflammation, which are easy to administer and which have no side effects.

This is the objective of the present invention, which, in order to meet it, aims at the use of particular bacteria of the human intestinal microbiota, namely bacteria of the Christensenellaceae family, preferably of the genus Christensenella. These bacteria can optionally be combined with other bacteria and, in particular, bacteria of the species Akkermansia muciniphila.

Bacteria of the Christensenellaceae family, in particular of the genus Christensenella, have already been studied and described. This is the case, in particular, with Christensenella minuta, Christensenella massiliensis and Christensenella timonensis. Christensenella minuta, in particular, was first described in 2012. In 2014, a study showed that it was the most heritable taxon in a cohort of British twins and that their presence is associated with a low body mass index. This correlation between Christensenella minuta and low body mass index was then observed in ten studies published since 2014 in geographically diverse populations.

The advantage of re-establishing a minimum population of Christensenella in individuals exhibiting an inflammatory intestinal disease linked to a deficiency of Christensenella in the intestinal microbiota was disclosed in application WO2016/156527. However, none of the aforementioned bacteria demonstrated a direct link with these pathologies in humans.

Furthermore, applications WO2018/162738 and WO2018/162726 teach that the use of particular bacteria of the Christensenellaceae family, belonging to species defined as being specifically distant from those of the genus Christensenella and, in particular, of the species Christensenella minuta and Christensenella timonensis, make it possible to fight, in particular, against overweight and obesity by intervening, for example, on the presence of visceral fat and on intestinal permeability.

Akkermansia muciniphila, belonging to the Verrucomicrobiaceae family and the genus Akkermansia, is a bacterium identified for the first time in 2004 and which represents approximately 1 to 3% by number of the total of the bacterial genera of the intestinal microbiota of healthy adults (Derrien et al., 2004, 54: 1469-1476; Derrien et al. Applied Environmental Microbiology 2008, 74, 1646-1648). The population in number of Akkermansia muciniphila has already been reported to be reduced in stool samples from obese individuals and patients with inflammatory gastrointestinal disease (Png, et al. Gastroenterol. 2010; 105: 2420-2428). Akkermansia muciniphila is associated with a healthier metabolic state and better clinical outcomes after calorie restriction intervention in overweight or even obese adults.

Surprisingly, and according to the invention, bacteria of the genus Christensenella, and in particular the species Christensenella minuta, Christensenella massiliensis and Christensenella timonensis, when administered to humans or animals, are capable of acting on the markers of inflammation at the origin of inflammatory diseases, in particular inflammatory gastrointestinal diseases and chronic inflammatory diseases; and cancers. In addition, the inventors, unexpectedly determined synergistic anti-inflammatory properties resulting from the use of a bacterium of the genus Christensenella in association with a bacterium of the genus Akkermansia, in particular Akkermansia muciniphila, a new generation probiotic (NGP) well known from the Verrucomicrobiaceae family.

Therefore, the invention relates to a bacterium of the genus Christensenella, for its use in the prevention and/or treatment of chronic inflammatory diseases and/or inflammatory gastrointestinal diseases and/or cancers in humans or animals.

Advantageously, such a bacterium, when it is administered to a human being or an animal exhibiting chronic inflammation, is capable of acting on the molecules produced in excess during chronic inflammation such as kynurenine, interleukin 6, interleukin 8, TNFalpha, interleukin 1b, lipocalins or fecal calprotectin, and/or by stimulating the production of the anti-inflammatory cytokine interleukin 10.

The invention also relates to compositions comprising at least one bacterium of the genus Christensenella, alone or in combination with at least one additional bacterium, preferably at least one additional bacterium of the genus Akkermansia, for its use in the prevention and/or the treatment of chronic inflammatory diseases and/or inflammatory gastrointestinal diseases and/or cancers in humans or animals.

Such a composition comprising, in a physiologically acceptable medium, at least one of these bacteria (at least one bacterium of the genus Christensenella alone or in combination with at least one additional bacterium of the genus Akkermansia) and/or their supernatant, is also targeted per se.

Other characteristics and advantages will emerge from the detailed specification and the figures of the invention which will follow.

### BRIEF DESCRIPTION OF THE DRAWINGS

FIG. 1 shows the production of interleukin-8 (IL-8) (in pg/mL) (ordinate) in HT-29 cells stimulated by TNF-α in the presence of strains of Christensenella minuta, Christensenella timonensis, Akkermansia muciniphila, Christensenella minuta+Akkermansia muciniphila and Christensenella timonensis+Akkermansia muciniphila (abscissa).

FIG. 2 shows the secretion of IL-8 by the intestinal cells of the HT29 line after co-incubation in the presence of supernatants of strains of Christensenella minuta (DSMZ: DSM22607) and stimulation by TNF-α. Dunn's nonparametric statistical test: *p<0.05; **p<0.01; ***p<0.001. IL-8: Interleukin 8; TNF-α: Tumor Necrosis Factor alpha. Means of 4 experiments repeated in triplicates.

FIG. 3 shows the measurement of the transepithelial resistance on intestinal cells of the Caco-2 line after co-incubation in the presence of Christensenella minuta bacteria (DSMZ: DSM22607) and after stimulation with TNF-α. Dunn's nonparametric statistical test: *p<0.05; **p<0.01; ***p<0.001. IL-8: Interleukin 8; TNF-α: Tumor Necrosis Factor alpha. Means of 3 experiments repeated in triplicates.

FIG. 4 shows the effects of Christensenella minuta (DSM 22607) on an inflammation model induced by DNBS. (A) Monitoring of the weight loss of the treated groups from the day of the injection (OJ). (B) Significant decrease in macroscopic scores indicating a reduction in the severity of inflammation following DSM 22607 and SASA treatments. Immunomodulatory effects of DSM22607 through the decrease in the activity of myeloperoxidase (C) and the increase of interleukin-10 (IL-10), an anti-inflammatory cytokine, in the spleen (D). Nonparametric ANOVA followed by Dunn's statistical test: *p<0.05; **p<0.01; ***p<0.001. DNBS: Dinitrobenzene sulfonic acid.

FIG. 5 demonstrates the generalization of the anti-inflammatory effects of the species of Christensenella minuta on a model of inflammation induced by DNBS. (A) Reduction in macroscopic scores indicating a reduction in the severity of inflammation following treatments with different species of Christensenella minuta. (B) Immunomodulatory effects of different species of Christensenella minuta through the decrease in myeloperoxidase activity (C). Nonparametric ANOVA followed by Dunn's statistical test: *p<0.05; **p<0.01; ***p<0.001. DNBS: Dinitrobenzene sulfonic acid.

FIG. 6 shows the effect of the supernatants of the strains of Christensenella minuta (DSMZ: DSM22607, C. minuta 1 and C. minuta 2) on the proliferation of HT-29 cells (adenocarcinoma of the colon). RLU: Relative Luminescence Unit. Dunnett's test where the control group is the GAM group (which represents the Gifu Anaerobic Broth culture medium). Code: *p<0.05, **p<0.01, ***p<0.001, ****p<0.0001.

### DEFINITIONS

Within the meaning of the invention, the term “bacteria” is also understood to mean the term “bacterial strains”. A bacterial strain should be understood as being a bacterium of a given strain belonging to a particular species, for example the species Christensenella minuta. Mention may be made, for example, of the bacterial strain or bacterium Christensenella minuta DSM22607. Thus, the two terms can be used interchangeably to designate one or several bacteria in the context of the invention.

Within the meaning of the invention, “hyperproduction of interleukin 6” is understood to mean an excessive production of interleukin 6 relative to the production in a healthy person or animal without inflammation.

Within the meaning of the invention, “hyperproduction of interleukin 8” is understood to mean an excessive production of interleukin 8 relative to the production in a healthy person or animal without inflammation.

Within the meaning of the invention, “under-production of interleukin 10” is understood to mean an insufficient production of interleukin 10 relative to the production in a healthy person or animal without inflammation.

Within the meaning of the invention, “chronic inflammatory disease” is understood to mean, in particular, any chronic disease whose slowly and gradually evolving pathophysiology is directly related to inflammation of autoimmune and/or auto-inflammatory origin.

Within the meaning of the invention, “inflammatory gastrointestinal disease” is understood to mean, in particular, an inflammatory bowel disease, more particularly an inflammatory bowel disease of the colon. Said colonic inflammatory bowel disease can, in particular, be selected from the group consisting of Crohn's disease (CD), ulcerative colitis (UC) and pouchitis, in particular CD and UC. CD and UC are two diseases characterized by inflammation of the lining of part of the digestive tract, linked to an overactive digestive immune system.

Within the meaning of the invention, “prevent” or “prevention” are understood to mean the reduction to a lesser degree of the risk or the probability of occurrence of a given phenomenon, i.e., in the context of the present invention, chronic inflammation, gastrointestinal inflammation and pathologies resulting therefrom such as cancer, in particular intestinal inflammation.

Within the meaning of the invention, “treat” or “treatment” are understood to mean a decrease in the progression of the disease, a stabilization, a reversal or regression, or even an interruption or inhibition of the progression of an inflammatory chronic disease, an inflammatory gastrointestinal disease, cancer. In the context of the invention, these terms also apply to one or more symptoms of said diseases of the present invention.

Within the meaning of the invention, “non-deficient in Christensenella” in the intestinal microbiota is understood to mean a human being or an animal whose bacteria of the genus Christensenella in their intestinal microbiota represents at least 0.01% by number of the total of the bacterial genera detected in their intestinal microbiota, in particular at least 0.1%, and preferably at least 0.5%. Collected in the stool, the abundance of Christensenella may, for example, be measured by a quantitative sequencing test, fluorescent in situ hybridization (FISH), qPCR (quantitative PCR) or by metagenome analyzes (relative quantification), well known to a person skilled in the art.

Within the meaning of the invention, “physiologically acceptable medium” is understood to mean a medium which is compatible with the organism of the individual to which said composition is to be administered. It may be, for example, a non-toxic solvent such as water. In particular, said medium is compatible with oral administration.

Within the meaning of the invention, “microbiota” is understood to mean all the microorganisms that have colonized an individual and with which he cohabits: bacteria for the most part, but also viruses, fungi, yeasts and protozoa. The composition of the microbiota differs depending on the colonized surfaces: we thus distinguish the cutaneous microbiota, the vaginal microbiota, the urinary microbiota, the respiratory microbiota, the ENT (otorhinolaryngology) microbiota and the intestinal microbiota, formerly called the intestinal flora, by far the most important with its 100,000 billion germs. Thus, “intestinal microbiota” is understood to mean all the microorganisms, in particular bacteria, which inhabit the intestine of a given individual.

According to an official definition of the World Health Organization (WHO), “body mass index” (BMI) is understood to mean the indicator of the health risks associated with excessive weight and insufficient weight. BMI is calculated by dividing an individual's weight (in kilograms) by their height (in meters) squared. A BMI value is associated with a specific body size according to the classification given by the WHO (https://www.who.int/features/factfiles/obesity/facts/en/).

“Effective amount”, “effective amount to”, “amount of X effective for” and the like, refer to an amount of an active ingredient which is effective to relieve, or to some extent reduce, one or more of the symptoms of the disease in need of treatment, or to delay the onset of clinical markers or symptoms of a disease which need prevention, when the compound is administered. Thus, the amount refers to an amount of the active ingredient which exhibits effects such as (i) reversing the rate of progression of a disease, (ii) inhibiting to some extent the progression of the disease, and/or, (iii) relieve to some extent (or, preferably, eliminating) one or more symptoms associated with the disease. The effectiveness of the amount may be determined empirically by testing the relevant compounds in known in vivo and in vitro model systems for a disease requiring treatment.

### DETAILED DESCRIPTION OF THE INVENTION

The subject matter of the invention is therefore the use, in the prevention and/or treatment of chronic inflammatory diseases and/or inflammatory gastrointestinal diseases and/or cancers, in particular cancers of an inflammatory nature, in human beings or animals, of at least one bacterium of the Christensenellaceae family, preferably of the genus Christensenella or a composition comprising at least one such bacterium and/or a culture supernatant of at least said bacterium of the genus Christensenella. The bacterium may be any bacterial strain of one of the species of the genus Christensenella. Preferably, said composition further comprises an additional bacterium of the Verrucomicrobiaceae family and/or a culture supernatant of said additional bacterium, in particular said bacterium being of the genus Akkermansia, more particularly of the Akkermansia muciniphila species.

Bacteria According to the Invention.

Therefore, the invention relates to a bacterium of the Christensenellaceae family, in particular of the genus Christensenella for its use in the prevention and/or treatment of inflammatory diseases, preferably of chronic inflammatory diseases and/or of inflammatory gastrointestinal diseases and/or cancer in humans or animals, in particular in people or animals with an inflammatory disease or cancer with hyperproduction of interleukin 6 and/or hyperproduction of interleukin 8 and/or underproduction of interleukin 10.

According to the invention, bacteria of the genus Christensenella, when administered to a human being or an animal exhibiting chronic inflammation, gastrointestinal inflammation or cancer, are capable of acting on the molecules produced in excess during chronic inflammation, gastrointestinal inflammation or cancer, in particular on interleukin 6, interleukin 8 and kynurenine, but also on TNFalpha, interleukin 1b, lipocalins or fecal calprotectin. Thus, the inventors have discovered that bacteria of the genus Christensenella have the unexpected ability to reduce in vitro and in vivo inflammation and cell proliferation, in particular gastrointestinal inflammation and more particularly chronic inflammation of the intestine. More particularly, the bacteria according to the present invention are capable of significantly reducing the production of pro-inflammatory molecules, such as interleukin 6 (IL-6) and interleukin 8 (IL-8).

Indeed, in inflammatory diseases and cancers which present an increase in one of these markers such as interleukin 6, interleukin 8, kynurenine, TNFalpha, interleukin 1b, lipocalin or calprotectin, their decrease is a sign of the reduction of the pro-inflammatory signal pathway, that is to say that the immune cells responsible for this production are less stimulated. As a result, the excessive immune reaction causing the inflammation is slowed down and the system gradually returns to normal.

According to one embodiment, the bacterium or bacteria useful according to the invention are administered to humans or animals in an amount effective for an action on at least one of these markers of inflammation, that is to say to decrease the production of at least one of these molecules in the body. According to a suitable embodiment, the bacterium or bacteria may be administered at a dose of 10⁷ to 10¹¹ colony-forming units (CFU) per day, regardless of the weight of the person or the animal, preferably a dose of 10⁹ to 10¹² CFU per day, more preferably a dose equal to 10⁹ CFU. In particular, the dose comprises at least one bacterium selected from Christensenella timonensis, Christensenella minuta, Christensenella massiliensis and/or their mixtures. Preferably, it is a single dose, i.e., administered all at once or a dose before each meal, i.e., three times a day.

Preferably, the bacterium of the Christensenellaceae family, in particular of the genus Christensenella, is useful in the treatment of at least one chronic inflammatory disease selected from chronic inflammatory diseases of the intestine, chronic inflammatory diseases of the liver, chronic inflammatory diseases of the pancreas, polyarthritis, atopic dermatitis, neuroinflammatory diseases of the chronic obstructive pulmonary disease. In particular, it has an effect in the prevention and/or treatment of at least one disease selected from Crohn's disease, ulcerative colitis, pouchitis, ulcerative colitis, celiac disease, autoimmune gastritis, hepatitis, nonalcoholic steatohepatitis, primary sclerosing cholangitis, pancreatitis, rheumatoid arthritis, psoriatic arthritis, psoriasis, eczema.

In addition, the bacterium of the Christensenellaceae family, in particular of the genus Christensenella, is useful in the treatment of at least one inflammatory gastrointestinal disease such as an inflammatory bowel disease, more particularly an inflammatory colonic bowel affection. For the purposes of the invention, the term “affection” is understood to mean a disease or pathology. Said inflammatory bowel affection in particular may be selected from the group consisting of Crohn's disease, ulcerative colitis and pouchitis, in particular from the group consisting of Crohn's disease and ulcerative colitis.

In addition, the bacterium of the Christensenellaceae family, in particular of the Christensenella genus, is useful in the treatment of a proliferative disease selected from lymphomas, glioblastomas, myelomas, leukemias, colorectal cancers, cancers of the breast, prostate, ovary, uterus, pancreas, lungs, liver, gallbladder and kidneys.

The bacterium or bacteria useful according to the invention are bacteria of the genus Christensenella. It may in particular be Christensenella massiliensis, Christensenella timonensis, Christensenella intestinihominis, and/or Christensenella minuta. According to a particularly suitable variant, it is Christensenella minuta.

These bacteria may be isolated from human stool, for example according to the protocols published by Morotomi et al. 2012 (Morotomi, M., Nagai, F. & Watanabe, Y. Description of Christensenella minuta gen. nov., Sp. nov., Isolated from human faeces, which forms a distinct branch in the order Clostridiales, and proposal of Christensenellaceae fam. nov. INTERNATIONAL JOURNAL OF SYSTEMATIC AND EVOLUTIONARY MICROBIOLOGY 62, 144-149 (2012)) and NDongo et al. 2016 (Ndongo, S., Dubourg, G., Khelaifia, S., Fournier, P. E. & Raoult, D. Christensenella timonensis, a new bacterial species isolated from the human gut. New Microbes and New Infections 13, 32-33 (2016)). These documents also describe the culturing methods of bacteria useful according to the invention. Optionally, said bacteria are available and accessible in the collection of microorganisms and cell cultures of German GmbH, in particular under filing numbers DSM 22607, DSM 102344, DSM 102800.

Composition According to the Invention.

The bacterium or bacteria useful according to the invention, for their use described above, are preferably administered in a composition, said composition comprising, in a physiologically acceptable medium, at least the useful bacterium or bacteria and/or their supernatant.

Thus, the invention also relates to a composition comprising at least one bacterium of the genus Christensenella in the prevention and/or treatment of chronic inflammatory diseases and/or inflammatory gastrointestinal diseases and/or cancers, in humans or animals, preferably in humans or animals exhibiting hyperproduction of interleukin 6.

In particular, the invention makes it possible to provide an effective amount of bacteria of the Christensenellaceae family, such as Christensenella massiliensis, Christensenella timonensis, Christensenella intestinihominis and/or Christensenella minuta, for use in the treatment of:
diseases associated with increased interleukin 6 such as an inflammatory disorder, allergic disorder, autoimmune disorder, transplant rejection disorder, atherosclerosis, osteoporosis, neuropathic pain, sepsis, Castleman's disease, a fibrotic condition, rheumatoid arthritis, juvenile rheumatoid arthritis, psoriatic arthritis, osteoarthritis, refractory rheumatoid arthritis, chronic non-rheumatoid arthritis or ankylosing spondylitis,
diseases associated with an increase in interleukin 8 such as atopic dermatitis, osteoarthritis, rheumatoid arthritis, inflammatory bowel disease, Crohn's disease, ulcerative colitis, stroke, septic shock, endotoxic shock, psoriasis, gram negative sepsis, toxic shock syndrome, renal reperfusion injury, glomerulonephritis, thrombosis, graft versus host reaction, allograft rejection, malaria, restenosis, angiogenesis, atherosclerosis, osteoporosis, gingivitis and unwanted release of hematopoietic stem cells, diseases caused by respiratory viruses, herpes virus and hepatitis viruses, meningitis, herpetic encephalitis, CNS vasculitis, traumatic brain injury, CNS tumors, subarachnoid hemorrhage, post-surgical trauma, cystic fibrosis, pruritus, interstitial pneumonia, hypersensitivity, crystalline arthritis, Lyme arthritis, progressive ossifying fibrodysplasia, acute and chronic pancreatitis, necrotizing enterocolitis, chronic sinusitis, uveitis, polymyositis, vasculitis, acne, gastric and duodenal ulcers, celiac disease, lupus, and/or
diseases associated with a decrease in interleukin 10 or which can be treated by increasing interleukin 10 such as autoimmune diseases, rejection of organ and bone marrow transplants, graft versus host disease, parasitic infections, granulomas, Crohn's disease, colitis, pancreatitis, inflammatory lung disease, inflammatory eye disease, atopic dermatitis and rhinitis.

According to one embodiment, the invention relates to a composition for its use in the treatment and/or prevention of chronic inflammatory diseases and/or inflammatory gastrointestinal diseases and/or cancers in a human being or an animal, said composition comprising, in a physiologically acceptable medium (i) at least one bacterium of the genus Christensenella, and/or (ii) a culture supernatant of at least one bacterium of the genus Christensenella.

The present invention also relates to a composition for its use in the treatment and/or prevention of an inflammatory gastrointestinal disease in an individual, said composition comprising, in a physiologically acceptable medium
(i) at least one bacterial strain selected from the group consisting of Christensenella timonensis, Christensenella minuta and/or their mixtures, and/or
(ii) a culture supernatant of at least any one of these bacterial strains and/or their mixtures.

According to any one of the preceding embodiments, the present composition may be administered to a human being suffering from chronic inflammatory diseases and/or inflammatory gastrointestinal diseases and/or cancers, said human being having a body mass index (BMI) less than 25, in particular said composition is administered to a human being suffering from inflammatory gastrointestinal diseases.

The term “BMI less than 25” denotes a BMI strictly less than 25, more particularly a BMI less than or equal to 24.9. According to any one of these embodiments, said human being may, in particular, have a BMI less than 25 and greater than or equal to 18.5.

Preferably, said composition according to the invention is characterized in that the bacterium of the genus Christensenella is selected from Christensenella massiliensis, Christensenella timonensis and/or Christensenella minuta and/or Christensenella intestinihominis and/or their mixtures. It may be, in particular, a mixture of Christensenella massiliensis and Christensenella timonensis or Christensenella massiliensis and Christensenella minuta or Christensenella massiliensis and Christensenella intestinihominis or Christensenella timonensis and Christensenella minuta or Christensenella timonensis and Christensenella intestinihominis or Christensenella minuta and Christensenella intestinihominis or Christensenella massiliensis, Christensenella timonensis and Christensenella minuta or Christensenella massiliensis, Christensenella timonensis and Christensenella intestinihominis or Christensenella timonensis and Christensenella minuta and Christensenella intestinihominis or Christensenella massiliensis, Christensenella minuta and Christensenella intestinihominis or Christensenella massiliensis, Christensenella timonensis and Christensenella minuta and Christensenella intestinihominis.

According to any one of the preceding embodiments, the composition may be used in an individual not deficient in Christensenella in the intestinal microbiota, in particular in a human being or an animal whose genus Christensenella represents at least 0.01% in number of the total of the bacterial genera of their intestinal microbiota.

The bacterium or bacteria are present in an effective amount in the composition providing an effect on chronic inflammatory diseases and/or inflammatory gastrointestinal diseases and/or cancers which are affecting the persons or animals treated. The term “person” is understood to mean an individual, in particular a human being or an animal.

Preferably, the composition useful according to any one of the preceding embodiments comprises 10⁶ to 10¹² colony-forming units (CFU) of bacteria of the genus Christensenella per daily dose of said composition to be administered.

Preferably, this corresponds to a daily dose of bacteria to be administered, regardless of the weight of the person or animal. Preferably, this dose is administered once per day. In particular, the useful composition comprises 10⁷ to 10¹¹ CFU bacteria of the genus Christensenella per daily dose to be administered, preferably 10⁹ CFU. In particular, said bacteria present in the composition may be of the same species, a mixture of species or a mixture of bacterial strains. The composition useful according to the invention may be in liquid form. It may, in particular, comprise bacteria of the Christensenellaceae family, in particular of the genus Christensenella and/or a culture medium for said bacteria which makes it possible to preserve them, such as, for example, preferably the anaerobic Columbia agar medium enriched with sheep blood, or an equivalent medium not containing any animal by-product. For the purposes of the present invention, the term “culture medium” means the culture supernatant in which the bacteria or bacterial strains have resided during their culture, or extracellular medium.

According to one variant, the composition useful according to the invention may be in solid form. In this case, the bacteria may be present in lyophilized form, and may also include excipients such as, for example, microcrystalline cellulose, lactose, sucrose, fructose, levulose, starches, stachyose, raffinose, amylum, calcium lactate, magnesium sulfate, sodium citrate, calcium stearate, polyvinylpyrrolidone, maltodextrin, galactooligosaccharides, fructooligosaccharides, pectins, beta-glucans, lactoglobulins, isomaltooligosaccharides, polydextroses, sorbitol and/or glycerol.

The compositions useful according to the invention may be in the form of powder, microencapsulated powder, gelatin capsule, capsule, tablet, lozenge, granules, emulsion, suspension, suppository, a beverage, a food, pharmaceutical or nutraceutical product, a food additive, dietary supplement or dairy product.

According to a particularly suitable embodiment, the compositions may be in a gastro-resistant form, such as a coated tablet containing microencapsulated bacteria. Said composition may thus be provided with a coating resistant to gastric juice in order to ensure that the bacteria of the invention included in said composition can pass through the damaged stomach. The release of the bacterium or bacteria can thus occur for the first time in the upper intestinal tract.

More particularly, the composition according to the invention may be included in a food supplement. A food supplement for oral administration may be present in capsules, hard capsules, soft capsules, tablets, sugar-coated tablets, pills, pastes, lozenges, gums, oral solutions or emulsions, a syrup or gel. A food supplement according to the present invention may further comprise a sweetener, a stabilizer, an antioxidant, an additive, a flavoring agent and/or a coloring agent. The formulation thereof is carried out by means of the usual methods for producing sugar-coated tablets, capsules, gels, hydrogels for controlled release, emulsions, tablets or capsules.

A composition according to the present invention may also be in the form of a nutritional composition. Such a nutritional composition according to the present invention is in the form of yogurt, cereal bar, breakfast cereal, dessert, frozen food, soup, pet food, liquid suspension, powder, tablet, gum or a piece of candy.

A composition according to the present invention may further comprise at least one ingredient selected from: antioxidants, fish oils, DHA, EPA, vitamins, minerals, phytonutrients, a protein, a lipid, probiotics and combinations thereof. The bacteria according to the invention, alone or included in the composition according to any one of the preceding embodiments, may be used in a live, semi-active, inactivated, or dead form, preferably in a living form. The semi-active, inactivated or dead form of said bacteria may be achieved, for example, by irradiation, thermal inactivation or lyophilization, in particular by heat, exposure to an appropriate pH, UV, gamma rays, X-rays or high pressure. Thus, the term “semi-active” designates a bacterium with low physiological activity whose ability to proliferate is reduced, temporarily or permanently. The term “inactivated” designates a bacterium which is no longer able, temporarily or permanently, to proliferate. The term “dead” designates a bacterium which is no longer able, definitively, to proliferate. Dead or inactivated bacteria may have intact or ruptured cell membranes. Thus, the term “inactivated” also designates the extracts and lysates of resultant bacteria.

A bacterium according to the invention, alone or in the composition according to the invention, may be used in whole form, i.e., essentially in its native form, or in the form of extracts or lysates comprising fractions and/or metabolites of this microorganism. Such a lysate may denote the whole of the lysate obtained by lysis or only a fraction thereof and may be prepared according to one of the conventional methods well known to a person skilled in the art such as, for example, a thermal shock, ultrasound, osmotic shock, or under mechanical stress, such as by centrifugation. They may all be live, semi-active, inactivated or dead. In particular, it may be a mixture of live, semi-active, inactivated and dead bacteria.

Preferably, at least some of the bacteria are live, in particular at least 50% (by number), even more preferably at least 90% (by number).

Thus, according to a suitable embodiment, at least 50% (in number) of the bacteria present in the composition useful according to the invention are living bacteria, preferably at least 90% (in number) are living bacteria, even more preferably are all living bacteria.

The storage conditions for liquid formulations according to the invention are in the form of frozen products maintained at −20° C. in sealed packets. For solid formulations, the storage conditions according to the invention comprise a capsule or a coating hermetic to light and to oxygen, maintained at an ambient temperature of between 15° C. and 40° C. and a humidity content of between 3% and 70%.

A composition according to any one of the preceding embodiments is provided for the digestive tract, in particular the intestine. Consequently, the bacteria useful according to the invention, and in particular the compositions comprising the same, may be administered by oral, topical, inhaled or rectal route, preferably rectal or intrarectal.

In particular, rectal or intrarectal administration is carried out in the form of a suppository, an enema or a foam.

The inventors have also determined, surprisingly, the ability of Christensenella strains to increase the anti-inflammatory properties of an Akkermansia strain and, in particular, of the Akkermansia muciniphila strain. Thus, the inventors demonstrated the existence of a synergistic effect of a combination of a Christensenella strain (or a culture supernatant of such a strain) and an Akkermansia strain (or a culture supernatant of such a strain) on the prevention and/or treatment of gastrointestinal inflammation, and in particular the synergistic effect of a combination of a Christensenella minuta and/or Christensenella timonensis strain (or culture supernatants of such strains) and an Akkermansia muciniphila strain (or a supernatant of such strain) on the prevention and/or treatment of gastrointestinal inflammation.

Thus, according to any one of the preceding embodiments, the composition may further comprise:
(i) at least one additional bacterium of the Verrucomicrobiaceae family; and/or
(ii) a culture supernatant of said additional bacterium.

Preferably, the additional bacterium of the Verrucomicrobiaceae family is a bacterium of the genus Akkermansia, more particularly of the species Akkermansia muciniphila. Within the meaning of the invention, a bacterium of the species “Akkermansia muciniphila” is also understood to mean the bacterium called “Akkermansia muciniphila”.

According to one embodiment, the bacterium and the additional bacterium as mentioned above are, independently of one another, in a live, semi-active, inactivated and/or dead form, and are in particular in a living form.

In certain embodiments, a composition according to the invention is suitable for the administration of a daily dose representing from 10′ to 10¹¹ colony forming units (CFU), preferably a daily dose equivalent to 10⁹ CFU, of a bacterium of the Verrucomicrobiaceae family, in particular of the genus Akkermansia, more particularly of an Akkermansia muciniphila bacterium.

In the case of the use of a culture supernatant of any one of the aforementioned bacteria, a composition according to the invention may, in particular, comprise a content of between 0.1 and 99% by weight, in particular from 10 to 90% by weight, in particular from 25 to 70% by weight and, more particularly, from 30 to 50% by weight, relative to the total weight of the composition. Thus, a composition according to any one of the aforementioned embodiments may comprise a content of between 0.1 and 99% by weight, in particular from 10 to 90%, in particular from 25 to 70% by weight, more particularly from 30 to 50% by weight of supernatant relative to the total weight of the composition.

The compositions useful according to the invention, in addition to the bacteria useful according to the invention, may comprise at least one probiotic, and/or at least one prebiotic.

The compositions useful according to the invention, in addition to the bacteria useful according to the invention, may comprise other compounds, such as:
at least one probiotic, and/or
at least one bacterium producing lactic acid which makes it possible to create an anaerobic environment favorable to Christensenellaceae, such as at least one bacterium selected from bacteria of the genus Lactobacillus spp., Bifidobacterium spp., Streptococcus spp. and/or at least one other organism promoting the anaerobic conditions necessary for the survival of Christensenellaceae, such as at least one yeast selected from Saccharomyces spp. or microorganisms of the Methanobacteriaceae family and/or
at least one bacterium associated with the ecosystem of Christensenellaceae because they facilitate their survival in the intestine, such as at least one bacterium selected from bacteria of the phylum Firmicutes, Bacteroidetes, Actinobacteria, Tenericutes, and Verrucomicrobia, and/or
at least one bacterium selected from bacteria of the order Clostridales, Verrucomicrobiales, Aeromonadales, Alteromonadales, ML615J-28, RF32, YS2, of the Clostridiaceae family, Lachnospiraceae, Ruminococcaceae, Bacteroidaceae, Enterococcaceae, Rikenellaceae, Dehalobacteriaceae, Veillonellaceae and/or
at least one bacterium selected from bacteria of the genus Faecalibacterium, Akkermansia, Eubacterium and Oscillospira such as for example Faecalibacterium prausnitzii, Akkermansia muciniphila, Eubacterium halii, Oscillospira guilliermondii, and/or
at least one prebiotic such as, for example, at least one prebiotic selected from galactooligosaccharides, fructooligosaccharides, inulins, arabinoxylans, beta-glucans, lactoglobulins and/or beta-caseins, and/or
at least one polyphenol such as, for example, at least one polyphenol selected from quercetin, kaempferol, resveratrol, flavones (such as luteolin), flavan-3-ols (such as catechins), flavanones (such as narinenin), isoflavones, anthocyanidins, proanthocyanidins, and/or
at least one mineral and/or at least one vitamin and/or at least one nutritional agent, and/or
at least one active pharmaceutical principle preferably selected from non-steroidal anti-inflammatory drugs, antibodies directed against pro-inflammatory targets (anti-TNFalpha), antirheumatic drugs, analgesics, antimicrobials, corticosteroids, anabolic steroids, antidiabetics, thyroid agents, antidiarrheals, cough suppressants, antiemetics, antiulcers, laxatives, anticoagulants, erythropoietin, immunoglobulins, immunosuppressants, growth hormones, hormonal drugs, estrogen receptor modulators, alkylating agents, antimetabolites, mitotic inhibitors, radiopharmaceuticals, anti-depressants, antipsychotics, anxiolytics, hypnotics, sympathomimetics, stimulants, donepezil, tacrine, asthma drugs, beta-agonists, inhaled steroids, leukotriene inhibitors, cromoglycates or cromoglicic acids, epinephrine, alpha dornase, cytokines, cytokine antagonists.

Lastly, the present invention relates to a composition comprising, in a physiologically acceptable medium:
(I) (a) at least one bacterium selected from the group consisting of Christensenella timonensis, Christensenella minuta, Christensenella massiliensis, Christensenella intestinihominis, and/or mixtures thereof;
and/or
(b) at least one culture supernatant of at least one bacterium selected from the group consisting of Christensenella timonensis, Christensenella minuta, Christensenella massiliensis, Christensenella intestinihominis, and/or mixtures thereof;
and
(II) (a) at least one additional bacterium of the genus Akkermansia, more particularly of the species Akkermansia muciniphila;
and/or
(b) at least one culture supernatant of at least one additional bacterium of the genus Akkermansia, more particularly of the species Akkermansia muciniphila.

Method of Prevention and/or Treatment.

According to another aspect, the present invention relates to a method for reducing and/or preventing inflammation in an individual in need thereof, comprising administering an effective amount of a bacterium of the Christensenellaceae family to said individual, in particular a bacterium of the species Christensenella.

The invention also relates to a method for reducing the level of IL-6 and/or stabilizing the level of IL-6 and/or preventing an increase in the level of IL-6 in an individual in need thereof, comprising administering an effective amount of a bacterium of the Christensenellaceae family to said individual, in particular a bacterium of the species Christensenella, preferably said individual exhibiting elevated serum levels of IL-6 before the treatment. In particular, said elevated serum IL-6 levels are greater than 5 pg/ml, greater than 10 pg/ml, greater than 20 pg/ml, greater than 30 pg/ml, greater than 40 pg/ml, greater than 50 pg/ml, greater than 70 pg/ml, greater than 90 pg/ml or greater than 100 pg/ml.

The invention also relates to a method for reducing the level of IL-8 and/or stabilizing the level of IL-8 and/or preventing an increase in the level of IL-8 in an individual in need thereof, comprising administering an effective amount of a bacterium of the Christensenellaceae family to said individual, in particular a bacterium of the species Christensenella, preferably, said individual exhibiting elevated serum IL-8 levels before the treatment. In particular, said elevated serum IL-8 levels are greater than 5 pg/ml, greater than 10 pg/ml, greater than 20 pg/ml, greater than 30 pg/ml, greater than 40 pg/ml, greater than 50 pg/ml, greater than 70 pg/ml, greater than 90 pg/ml or greater than 100 pg/ml. The invention also relates to a method for increasing the level of IL-10 and/or stabilizing the level of IL-10 and/or preventing a decrease in the level of IL-10 in an individual in need thereof, comprising administering an effective amount of a bacterium of the Christensenellaceae family to said individual, in particular a bacterium of the species Christensenella, preferably, said individual exhibiting low serum IL-10 levels before the treatment. In particular, said low serum IL-10 levels are less than 15 pg/ml, less than 10 pg/ml or less than 5 pg/ml.

In addition, the invention relates to a method for reducing the level of kynurenine and/or stabilizing the level of kynurenine and/or preventing an increase in the level of kynurenine in an individual in need thereof, comprising the administration of an effective amount of a bacterium of the Christensenellaceae family to said individual, in particular a bacterium of the species Christensenella, preferably, said individual exhibiting high levels of fecal kynurenine before the treatment.

The invention also relates to a method of treating and/or preventing cancer associated with inflammation in an individual in need thereof, comprising administering an effective amount of the Christensenellaceae bacteria to said individual.

The method according to the present invention makes it possible to reduce the level of at least one of the markers of inflammation mentioned above in said individual, said marker being preferably reduced in serum, blood or stool.

Preferably, the present methods are implemented in an individual suffering from one or more diseases described above in the specification, said individual preferably being a human being.

Preferably, the bacterium Christensenella comprises a 16S ribosomal RNA gene exhibiting a sequence identity of at least 50%, at least 60%, at least 70%, at least 80% or, preferably at least 90% with a 16S ribosomal RNA gene from Christensenella minuta, Christensenella massiliensis, or Christensenella timonensis, more preferably at least 97% sequence identity with a sequence selected from SEQ ID NO: 1, 2, 3, 4, 5 or 6.

SEQ ID NO: 1 is a sequence of the 16S ribosomal RNA gene of the bacterium Christensenella minuta DSM 22607, which was isolated from a human fecal sample. The C. minuta 16S rRNA sequence is identified by the Gen Bank accession number AB490809. SEQ ID NO: 2 is part of the 16S ribosomal RNA gene sequence from the bacterium Christensenella minuta DSM 22607. Additional 16S sequences for bacteria of the genus Christensenella, such as SEQ ID NOs: 3-6 (OTU numbers 701845, 177179, 1146771, and 361793 in the Greengenes database, respectively), are provided. The identity of each sequence with respect to SEQ ID NO: 1 is as follows: SEQ ID NO: 3, 93% identity with SEQ ID NO: 1; SEQ ID NO: 4, 95% identity with SEQ ID NO: 1; SEQ ID NO: 5, 97% identity with SEQ ID NO: 1; SEQ ID NO: 6, 95% identity with SEQ ID NO: 1.

In particular, said methods comprise the administration of at least one bacterium or composition according to the present invention, characterized by any of the embodiments described above, such as the mixture or not of the bacteria, the quantity administered, the amount of viable bacteria, the additional components, for example active pharmaceutical ingredients, prebiotics or probiotics, vitamins, minerals, nutritional agents, other microorganisms, the particular formulation of said composition, for example powder, capsule, etc.

Understandably, the present methods include oral, inhaled, topical, rectal or intrarectal administration in a sufficient amount.

The invention is now illustrated by examples of bacteria useful according to the invention, methods of culturing these bacteria, examples of compositions containing the same, and test results demonstrating the effectiveness of the invention which are shown for illustration purposes only.

### EXAMPLES

### Example 1: Christensenella minuta

Christensenella minuta can be cultured according to the operating protocol described below.

1/ Dissolve a dehydrated RCM (Reinforced Clostridial Medium) in distilled water
2/ Add 0.5 ml/L of resazurin-Na solution (0.1% w/v)
3/ Bring to a boil and cool to room temperature while injecting a gas mixture of 80% N₂ and 20% CO₂
4/ Spread the medium under the same gaseous atmosphere in anoxic Hungate-type tubes or in vials of serum then autoclave
5/ Before use, add 1.0 g of sodium carbonate per liter from a sterile anoxic stock solution prepared with a gas mixture of 80% N₂ and 20% CO₂
6/ Check the pH of the medium after autoclaving and adjust the pH between 7.3 and 7.5, using a sterile anoxic stock solution of sodium bicarbonate (5% w/v) prepared in a gaseous atmosphere at 80% N₂ and 20% CO₂.

### Example 2: Christensenella massiliensis

Christensenella massiliensis can be cultured according to the operating protocol described below.

1/ Prepare a carboxymethylcellulose (N₂/CO₂) medium by following the instructions below provided by DSMZ (Deutsche Sammlung von Mikroorganismen and Zellkulturen).

TABLE 1

| | | |
|---|---|---|
| Casitone | 30.0 | g |
| Yeast extract | 5.0 | g |
| K₂HPO₄ | 5.0 | g |
| Na-resazurin solution (0.1% w/v) | 0.5 | ml |
| L-Cysteine-HCl × H₂O | 0.5 | g |
| D-Glucose | 4.0 | g |
| Cellobiose | 1.0 | g |
| Maltose | 1.0 | g |
| Minced meat (fat free) | 500.0 | g |
| NaOH 1N | 25.0 | ml |
| Distilled water | 1000 | ml |
| Starch (soluble) | 1.0 | g |
| Na₂CO₃ | 1.5 | g |
| Meat filtrate (see Table 2) | 1000 | ml |

2/ Dissolve the various constituents listed in the table above, with the exception of cysteine, carbohydrates and carbonate.
3/ Boil the medium for 1 min, then let it cool to room temperature under a gaseous atmosphere at 80% N₂ and 20% CO₂.
4/ Add 0.5 g/L L-cysteine-HCl×H₂O and pour it under the same gaseous atmosphere into Hungate-type tubes (for strains requiring meat particles, introduce these first into the tube, use 1 part meat particles for 4 or 5 parts liquid).

### 5/ Autoclave at 121° C. for 20 min.

6/ After autoclaving, add glucose, cellobiose, maltose and starch from sterile anoxic stock solutions prepared with 100% N₂ gas and carbonate from a sterile anoxic stock solution prepared under gas mixtures at 80% N₂ and 20% CO₂.
7/ Adjust the pH of the medium to 7, if necessary.

Preparation of the Meat Filtrate:

TABLE 2
Use lean beef or horse meat.
Remove fat and connective tissue before chopping.
Mix the meat, water and NaOH, then boil for 15 min while stirring.
Cool to room temperature, remove the fat from the surface and strain,
retaining the meat particles and the filtrate.
Add water to the filtrate to a final volume of 1000.0 ml.
Bacteria should be cultured in anaerobic condition at 37° C.

### Example 3: Christensenella timonensis

Christensenella timonensis can be cultured according to the same operating protocol as that described in Example 2 for Christensenella massiliensis.

### Example 4: Specific Strains According to the Invention

All the strains described below were obtained from the German DSMZ collection (Leibnitz DSMZ Institute). Christensenella minuta DSM 22607, Christensenella timonensis DSM 102800 and Akkermansia muciniphila DSM 22959 were cultured at 37° C. in YBHI medium [brain-heart perfusion medium supplemented with 0.5% yeast extract (Difco)] supplemented with cellobiose (1 mg/ml; Sigma), maltose (1 mg/ml; Sigma), cysteine (0.5 mg/ml; Sigma) and mucin (1 mg/ml; Sigma) in an anaerobic chamber filled with N₂=85%, CO₂=10% and H₂=5%.

### Example 5: Useful Composition Based on Supernatants

An example of a composition useful according to the invention is a composition comprising between 0.1 and 95% culture supernatant of the strains Christensenella minuta DSM 22607, Christensenella timonensis DSM 102800 and Akkermansia muciniphila DSM 22959.

### Example 6: Composition in Liquid Form Useful According to the Invention

An example of a composition in liquid form useful according to the invention is a composition comprising Christensenella minuta 10⁹ CFU/ml in the anaerobic RCM culture medium described above, modified to contain no animal by-product and enriched in 5% glycerol.

The composition of Example 6, obtained from an RCB (Research Cell Bank), is prepared from 10¹⁰ CFU/ml Christensenella minuta then stored frozen at −20° C. in a packet sealed off from oxygen. The frozen composition must be warmed to room temperature until it returns to a liquid form before use.

### Example 7: Composition in Solid Form Useful According to the Invention

An example of a composition in lyophilized form useful according to the invention may be obtained by lyophilization of the composition of Example 6 in the frozen state.

Test Results Demonstrating the Effect of the Invention

1. Demonstration of the In Vitro Anti-Inflammatory Effect of Christensenella Bacteria (Kynurenine).

The objective of this study is to demonstrate the in vitro anti-inflammatory effect of Christensenella bacteria according to the invention. The demonstration of this effect was carried out on kynurenine, a known marker of inflammation. Human faeces were collected and cultured in a fermenter. The abundance of Christensenella spp. was correlated with the abundance of kynurenine present in the fermenter.

The operating protocol of the study is described below.

1/ Fermentation Protocol from Faeces of Human Origin Containing Christensenella Spp.:
Donors should not have taken antibiotics during the six months preceding the experiment and have no history of gastrointestinal disorders. The donors were between 18 and 60 years old.
Collection of fresh samples of their faeces is obtained in sterile plastic containers, kept in anaerobic vials containing an Oxoid™ AnaeroGen™ 2.5 L sachet (O₂<0.1%; CO₂: 7-15%). These samples were brought to the laboratory within two hours of production.
The faeces samples were diluted ⅕ (weight/volume) in phosphate buffered saline (1M) (PBS), pH 7.4. The suspension was homogenized in a stomacher for 120 seconds.
Basic nutrient medium: the basic nutrient medium was prepared from 2 g/L of soybean tryptone broth, 2 g/L yeast extract, 0.1 g/L NaCl, 0.04 g/L K₂HPO₄, 0.01 g/L MgSO₃.7H₂O, 0.01 g/L CaCl₂.6H₂O, 2 g/L NaHCO₃, 0.5 g/L L-cystine HCl, 2 ml/L tween 80, 10 μl/L vitamin.K1, 0.05 g/L heme, 0.05 g/L bile salts, 4 ml/L resazurin (pH7).
Fermentation in a biofermenter: The 20 ml biofermenters contained 18 ml of basic nutrient medium, autoclaved (121° C. for 15 minutes) and poured aseptically into sterile biofermenters. This system was allowed to stand overnight with oxygen-free nitrogen bubbling through the medium at a rate of 2 ml/min. The pH was maintained between 6.7 and 6.9 using HCl or NaOH (0.5M). The temperature of each biofermenter was controlled at 37° C. and the contents of the container homogenized with a magnetic mixer.
A mixture of predigested proteins (0.35 g) was added to the containers before inoculation with 2 ml fecal inoculum at TO. The predigested proteins were obtained according to the gastrointestinal digestion protocol adapted from that of Versantvoort et al. (2005).
The samples were collected before fermentation (TO) and after 48 hours of fermentation (T48), and frozen at −80° C. until analyzes were carried out.

2/ Quantification of Kynurenine
50 μL of samples collected and stored at −80° C. were mixed with 20 μL of Milli-Q water containing internal standards.
The mixture was mixed and filtered through a 5-kDa cutoff filter to remove macromolecules.
The metabolites were detected by capillary electrophoresis-time-of-flight mass spectrometry (CE-TOFMS) analyzes. The peak detection limit was determined based on the signal-to-noise ratio, S/N=3.

Relative peak area=(peak area of a metabolite):(peak area of internal standard x amount of sample).

3/ Quantification of Christensenella spp.
The DNA contained in the samples was extracted using the NucleoSpin® 96 Soil kit from Macherey-Nagel according to the manufacturer's instructions.
The total extracted DNA was then fragmented randomly into fragments of 350 bp then used to construct a library using the NEBNext Ultra II kit from New England Biolabs according to the manufacturer's instructions.
The library was then sequenced using 2×150 bp paired-end sequencing on an Illumina HiSeq platform.
The abundance of bacteria was measured by creating a catalog of metagenomic species (MGS) from a reference catalog containing 22M genes. These MGSs were then associated at an appropriate taxonomic level. In the case of Christensenella, these were detected at the genus-level and are therefore referenced in this experiment by Christensenella spp.

The relative amount of kynurenine (relative to the total amount measured) and the relative abundance of Christensenella spp. (relative to the total quantity measured) were analyzed and correlated, obtaining a linear regression of R=−0.44 (n=18). The results are shown in Table 3.

TABLE 3

| | Relative abundance of | Relative amount of |
|---|---|---|
| Samples | Christensenella spp. (×10⁻²) | kynurenine (×10⁻⁵) |
| V1 | 7.55 | 0 |
| V2 | 3.18 | 0 |
| V3 | 8.19 | 0 |
| V4 | 2.63 | 3.46 |
| V5 | 1.26 | 3.67 |
| V6 | 2.87 | 0 |
| V7 | 7.20 | 8.18 |
| V8 | 2.91 | 4.58 |
| V9 | 6.32 | 0 |
| V10 | 1.21 | 13.00 |
| V11 | 4.23 | 14.19 |
| V12 | 1.49 | 8.36 |
| V13 | 9.83 | 0 |
| V14 | 4.12 | 2.87 |
| V15 | 6.45 | 0 |
| V16 | 2.02 | 5.30 |
| V17 | 5.23 | 4.76 |
| V18 | 7.57 | 2.92 |

2. Demonstration of the In Vitro Anti-Inflammatory Effect of Christensenella Bacteria (11-8)

The objective of this study is to demonstrate the in vitro anti-inflammatory effect of bacteria according to Example 4. The demonstration was carried out on one of the markers of inflammation: interleukin 8 (IL-8).

The study is carried out on HT-29 cells obtained as follows: HT-29 (ATCC HTB-38) human colon epithelial cells (LGC Standards) were cultured in Dulbecco's

Modified Eagle's minimum essential medium (DMEM) (Sigma-Aldrich) supplemented with 10% (w/v) thermo-activated fetal bovine serum (FBS) (GibcoBRL, Eragny, France) and penicillin G/streptomycin (5000 IU/ml, 5000 μg/ml) (Sigma-Aldrich). Cell cultures were incubated in 25-cm² tissue culture flasks (Nunc, Roskilde, Denmark) at 37° C. in a 5% (v/v) CO₂ atmosphere until confluence is reached. The operating protocol of the study is described below.

The single cultures and the cocultures composed of the combinations between the listed strains are prepared at time 0. At 24 hours, the bacterial samples are centrifuged for 10 minutes at 6000 rpm. The supernatants thus obtained are stored at −80° C. in order to continue the analysis of the in vitro immunomodulatory properties.

Anti-inflammatory tests were carried out according to the procedure described by Kechaou et al., 2012 (Kechaou N et al.: Appl Environ Microbiol 2012, 79:1491-1499). Specifically, 50,000 HeLa or HT-29 cells per well were seeded in 24-well culture plates (Nunc). Twenty-four hours before the bacterial contact, the culture medium was replaced with a medium containing 5% FBS. The experiments were undertaken on the 7th day after seeding, when the cells were at confluence (1.83×10⁶ cells/well). Twenty-four hours before the bacterial coculture (day 6), the culture medium was replaced with a medium containing 5% heat-inactivated FBS and 1% glutamine. On the day of the coculture, 10% bacterial supernatant was added to the DMEM in a total volume of 500 μl. Cells were simultaneously stimulated with human TNF-α (5 ng/ml; Peprotech, N.J.) for 6 hours at 37° C. in 10% CO₂. All samples were analyzed in triplicate. After co-incubation, cell supernatants were collected and stored at −80° C. until ELISA (Biolegend, San Diego, Calif.) performed further analysis of interleukin-8 (IL-8). The experiments were carried out at least in triplicate.

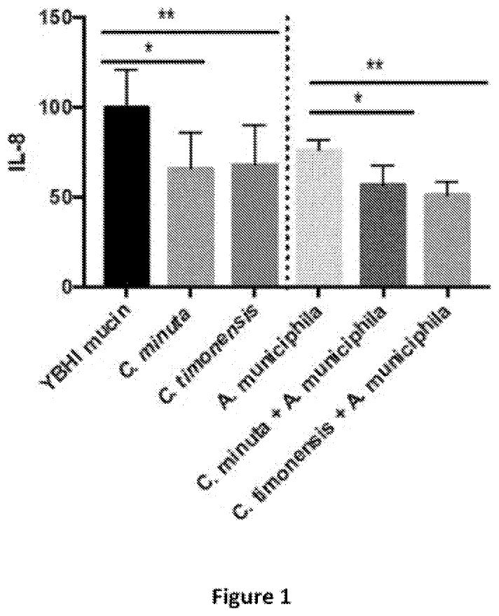

FIG. 1 shows the production of IL-8 in HT-29 cells stimulated by TNF-α in the presence of Christensenella minuta, Christensenella timonensis, Akkermansia muciniphila, Christensenella minuta+Akkermansia muciniphila and Christensenella timonensis+Akkermansia muciniphila. The results are expressed in IL-8 (pg/mL) and were normalized using as reference value IL-8 produced after co-incubation with PBS as negative control. The results were analyzed using Kruskal Wallis test followed by Dunn's multiple comparison test (*p<0.05; **p<0.01).

GraphPad software (GraphPad Software, La Jolla, Calif., USA) was used for the statistical analysis. The results are presented in the form of a boxplot (min. to max.). Comparisons were made with the Kruskal-Wallis nonparametric test followed by a Dunn's multiple comparison. A p-value less than 0.05 was considered significant.

The results are described below:

The reference strain Akkermansia muciniphila DSM 22959 is well known for its immunomodulatory properties and, more particularly, for its anti-inflammatory effects (Derrien M, Belzer C, de Vos WM. Microb Pathog. 2017 May; 106:171-181. doi: 10.1016/j.micpath.2016.02.005. Epub 2016 Feb. 11). To determine whether the strains of Christensenella minuta DSM 22607 and Christensenella timonensis DSM 102800 are capable of modulating the immune response, the inventors tested in vitro the immunomodulatory properties of the supernatants of the strains in a model based on the ability to block the production of IL-8 (a pro-inflammatory cytokine) induced by TNF-α stimulation in HT-29 colon epithelial cells.

Thus, the inventors observed that the two strains are capable of blocking the production of IL-8 in the same way as our positive control (Akkermansia muciniphila DSMZ 22959) (FIG. 1).

Christensenella minuta DSM 22607 and Christensenella timonensis DSM 102800 increase the anti-inflammatory properties of Akkermansia muciniphila DSMZ 22959 in vitro.

In order to test whether the members of Christensenella were able to increase the anti-inflammatory properties of other members of the intestinal microbiota, cocultures with Akkermansia muciniphila were carried out. As also shown in FIG. 1, Christensenella minuta DSM 22607 and Christensenella timonensis DSM 102800 were able to increase the anti-inflammatory properties of Akkermansia muciniphila DSMZ 22959 in a statistically significant manner.

3. Demonstration of the In Vitro Anti-Inflammatory Effect of Christensenella Bacteria (11-8).

The objective of this study is to demonstrate the anti-inflammatory effect in vitro of bacteria according to the invention. The demonstration was carried out on one of the markers of inflammation: interleukin-8.

The operating protocol for the study is described below.

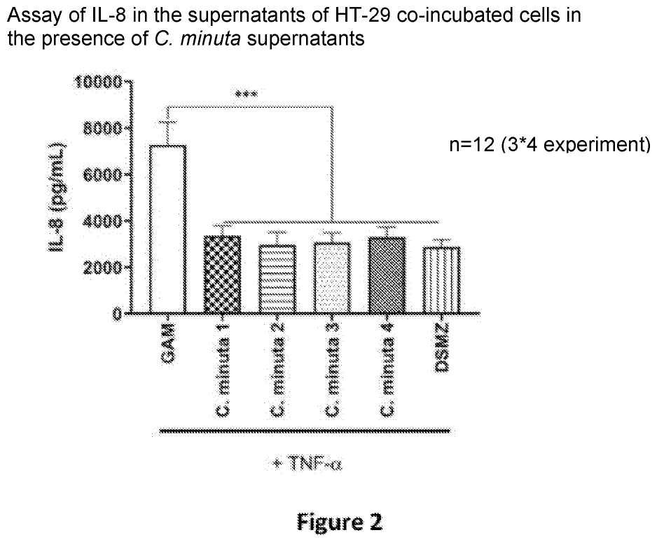

When the HT-29 cells are at confluence (approximately 7 days after seeding), the complete medium (DMEM Glutamax+10% FCS) is then removed in order to be replaced by medium containing 5% FCS (DO). On D+1, the medium is removed. Two media (DMEM Glutamax+5% FCS+/−TNF-α) are prepared. 450 μL/well of one of the two media are then added, followed by 50 μl of the supernatants to be tested. After 6 hours of co-incubation, the supernatants are recovered and stored at −80° C. IL-8 chemokine ELISA assays are then carried out (ELISA MAX Deluxe set Human IL-8 from Biolegend). FIG. 2 shows the assay of the chemokine IL-8 secreted by HT-29 cells (colon adenocarcinomas) in an inflammatory state induced by TNF-α. Different bacterial supernatants (C. minuta 1, C. minuta 2, C. minuta 3, C. minuta 4 and DSMZ, corresponding to strain DSM22607) belonging to bacteria of the species Christensenella minuta were tested in order to evaluate their immunomodulatory capacity. The bacterial culture medium (GAM) represents the control making it possible to assess the effects of the supernatants. The experiment was carried out four times in triplicate.

All the strains of Christensenella minuta tested have an anti-inflammatory immunomodulatory effect (reduction of more than 50% in the level of IL-8 secreted), which makes it possible to confirm the anti-inflammatory potential of Christensenella.

4. Demonstration of the Effect of Christensenella Bacteria on the Permeability of the Intestinal Epithelial Membrane.

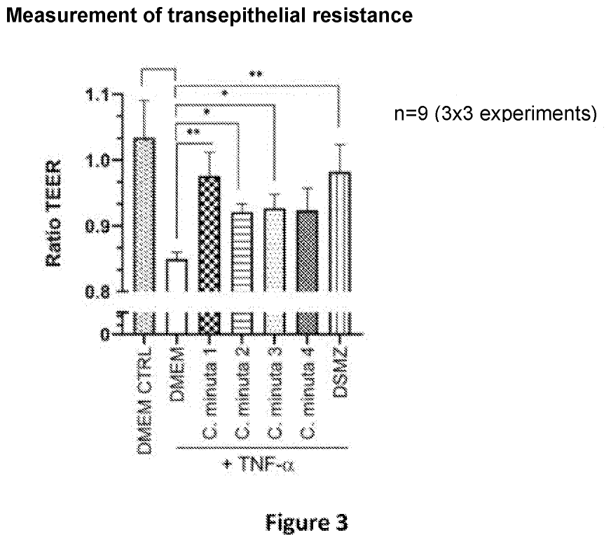

FIG. 3 represents the measurement of the transepithelial electrical resistance taken on Caco-2 epithelial cells. The latter establish junctions (called tight junctions) between them by differentiating, allowing to maintain the integrity of this barrier created by the cells, mimicking the intestinal barrier present in our digestive tract.

The addition of the cytokine TNF-α (Tumor Necrosis Factor) disrupts these tight junctions then increases membrane permeability, promoting, for example, inflammatory or infectious diseases and thus reducing the TEER ratio (DMEM+TNF-α control group). The “DMEM” control group represents the wells where only the cell culture medium was present, without TNF-α or bacteria. Transepithelial electrical resistance (TEER) is a method for quantifying the integrity of the barrier of epithelial tissue by measuring the electrical resistance therethrough. TEER measurements are performed by placing electrodes at both poles of a layer of epithelial cells that have been cultured on a semi-permeable membrane. The electrodes apply an alternating current allowing the measurement of the electrical resistance through the layer of epithelial cells.

The TEER ratio is obtained by the following calculation:

$$TEER ratio = \frac{TEER treatment t 24 / TEER treatment t 0}{TEER control t 24 / TEER control t 0}$$

7 days after seeding of Caco-2 cells (intestinal epithelial cells) in Transwell plates, the TEER is measured using an automated machine (REMS). The values are recorded and represent our starting value (TO). The cells are then co-incubated in the presence of bacteria in (MOI 1/40) for 3 hours, the time for the latter to adhere to the cells. The cells are then challenged with TNF-α inducing a disruption of the epithelial cell layer, in particular at the tight junctions. 24 hours (T24) after adding the bacteria, the TEER is measured again.

This figure shows that the Christensenella minuta bacteria influence the integrity of the intestinal barrier by improving transepithelial resistance, indicating a potential effect on tight junction proteins and a decrease in intestinal permeability. Therefore, the Christensenella minuta bacteria make it possible to restore the resistance of the epithelial membrane.

5. Demonstration of the In Vivo Anti-Inflammatory Effect of Christensenella Bacteria

The objective of this study is to demonstrate the in vivo anti-inflammatory effect on mice, of bacteria according to the invention. The demonstration was carried out on major markers of inflammation, two cytokines: IL-6 (interleukin 6) and TNF-α (Tumor Necrosis Factor-alpha). The study was carried out as a blind study: the experimenters did not know the treatments so that their previous knowledge did not influence the result of the study in any way.

The operating protocol is described below.
Four-week old C57BL/6 male mice were purchased from Charles River (St Germain sur I'Arbresle, France).
The mice had a one-week period of acclimatization on the diet based on a standard diet (“chow diet (SafeA04)”) before starting the study.
On the first day (DO), the animals were subjected to a diet rich in fat (45% kcal “Research Diet D12451”) and distributed randomly into 2 groups (n=5 per group), each receiving either a solution of 150 μL of Christensenella minuta (10⁹ CFU/mL), i.e., a control solution consisting of the culture medium used for the growth of bacteria for 12 weeks. The animals were provided with food and water at will throughout the duration of the study and were kept in a room at controlled temperature (22.0±2.0° C.) and humidity (40-50%) and on a cycle of 12 hours of light (8 a.m. to 8 p.m.)/12 hours of darkness.
At the end of the experiment (D85), the last blood samples were taken from the anesthetized animals (with a mixture of ketamine/xylazine 80/10 mg/kg). Blood samples were collected in centrifuge tubes pre-filled with heparin sulfate (200 IU/ml blood). The plasma was separated by centrifugation (3500 rpm, 15 min., 4° C.), collected and then stored at −80° C. until analysis.
The plasma levels of cytokines IL-6 and TNF-α were determined using the “Invitrogen® Luminex® Cytokine Mouse Magnetic 10-Plex” ELISA assay kit and according to the manufacturer's instructions. The measurements were carried out by VEBIO (Arcueil, France).

The assay results (presented in Table 4) of the pro-inflammatory cytokines TNF-α and IL-6 after 85 days of a high-fat diet at 45% (cal) show that a bacterium of the Christensenellaceae family strongly limits inflammation.

TABLE 4

| | | | Mean | | Mean |
|---|---|---|---|---|---|
| | | TNF-α | TNF-α +/− | IL-6 | IL-6 +/− |
| Diet | Mouse | (pg/mL) | SEM | (pg/mL) | SEM |
| HFD45% + | C9-SM | | — | 62.48 | 44.61 +/− |
| C. minuta | C9-OG | | | 17.00 | 11.40 |
| | C15-SM | | | 52.17 | |
| | C15-OG | | | 72.73 | |
| | C15-OD | | | 18.68 | |
| HFD45% + | C12-SM | 2.93 | 3.53 +/− | 89.71 | 117.25 +/− |
| control | C12-OG | | 0.36 | 144.76 | 28.57 |
| | C12-OD | 4.19 | | 39.96 | |
| | C14-SM | 3.49 | | 101.85 | |
| | C14-OG | 1.87 | | 209.96 | |

6. Demonstration of the In Vivo Anti-Inflammatory Effect of Christensenella Bacteria

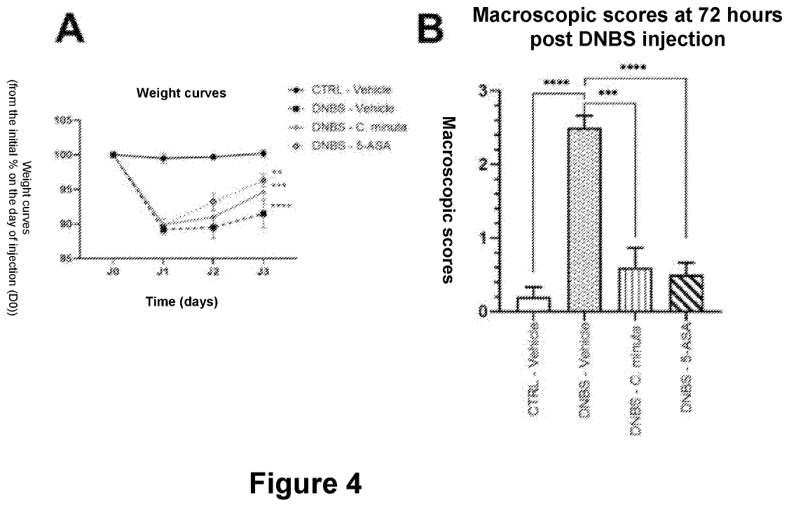

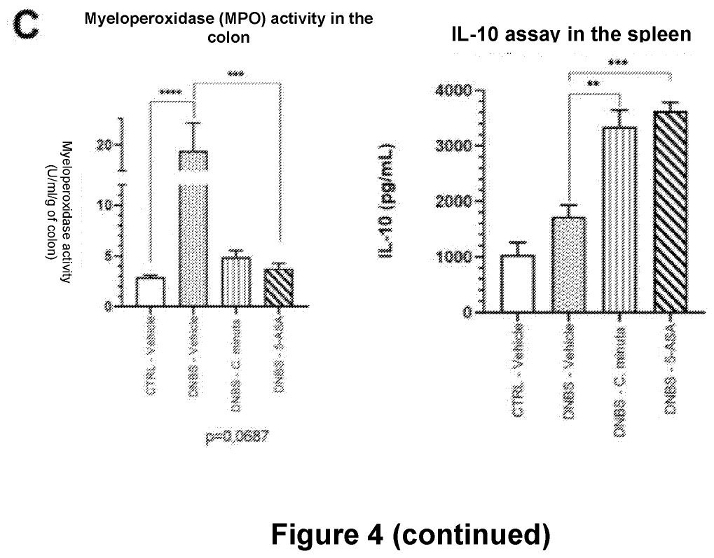

As soon as they arrive, the animals are put in a cage (n=5 per cage) and start an acclimatization period in order to allow the mice to recover from transport and reduce stress by getting used to this new environment. After one week, gavage of all groups (except the 5-ASA control group, pharmacological anti-inflammatory control) as well as weight monitoring begin. The control mice (ctlr-vehicle and DNBS-vehicle) are gavaged with 150 μl of PBS/16% glycerol. The DNBS-Christensenella minuta (C. minuta) mice are gavaged with 150 μl of C. minuta DSM 22607 at 10¹⁰ CFU/mL resuspended in PBS/16% glycerol. After two weeks, inflammation is induced by means of a chemical called DNBS (dinitrobenzensulfate). The latter is injected directly into the colon of the mouse by a probe introduced into the rectum. DNBS is dissolved in 30% ethanol, making it possible to weaken the intestinal barrier to facilitate the entry of DNBS into the tissue, thus triggering the immune system and therefore inflammation. The gavages continue during this period, and those of the 5-ASA group begin on the day of this injection at 150 μL (solution of 5-ASA prepared every morning, at 2 mg/mouse resuspended in PBS). Euthanasia takes place 3 days after the injection. The so-called “recovery” period is therefore short, but necessary to be able to observe differences with the DNBS control group. FIG. 4 shows the various parameters evaluated during a test on an inflammatory model induced by DNBS.

In FIG. 4A, the monitoring of the weight loss was observed from the day of the injection, and therefore the recovery period (D1, D2 and D3) which follows. The inventors have thus observed an effect on the recovery which seems to be faster after the administration of the treatment.

In FIG. 4B, the macroscopic scores show the level of severity of the disease, indicate that following the treatments with DSM 22607, a significant decrease in the score was observed, also observed with the pharmacological anti-inflammatory agent, 5-ASA (5-aminosalicylic acid used as a comparative positive control).

FIGS. 4C and 4D show the immunomodulatory potential of DSM 22607, in particular by reducing the presence of neutrophils in the colon, evaluated by monitoring the activity of myeloperoxidase. In addition, the increase in interleukin-10 in the spleen (FIG. 4D) shows the anti-inflammatory effects associated with Christensenella minuta.

7. Demonstration of the In Vivo Anti-Inflammatory Effect of Christensenella Bacteria

Three days after the injection, the animals are euthanized. The change in the weight of the mice is measured by taking the weight of the mice daily. During the sacrifice, the colon is opened and cleaned in order to carry out the macroscopic score. The latter is based on the presence of blood in the tissue (hyperemia), the presence of ulcer, the intestinal transit (for example if the mouse was constipated) and the thickening of the colon induced by DNBS.

A piece of colon is also recovered in order to assay the activity of myeloperoxidase (MPO). This enzyme is present in neutrophils, markers of inflammation. It also participates in the production of reactive oxygen species (ROS). During this test, after weighing the colon to normalize the results according to the size of the sample, a calorimetric reaction takes place to measure the activity of this enzyme.

The spleen is also recovered during the sacrifice (peripheral lymphoid organ) and the cells of the innate and adaptive immune response are stimulated in order to assay the secreted cytokines such as IL-10, anti-inflammatory cytokines.

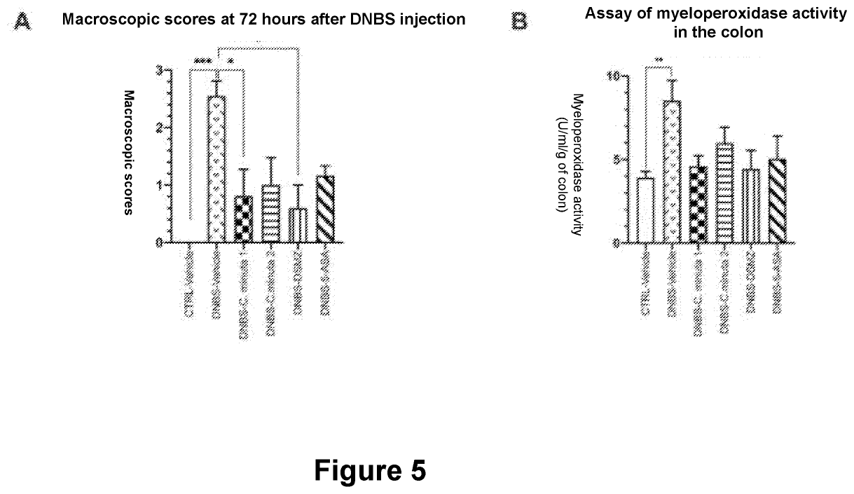

FIG. 5 shows the effects of other strains of Christensenella minuta during a repetition of the experiment presented in FIG. 4. The experimental design and the methods remain similar. This experience teaches us that all the strains of Christensenella minuta tested have the same efficacy in reducing macroscopic scores following intrarectal injection of DNBS. Likewise, all the strains tested show an anti-inflammatory effect which results in a reduction in MPO activity.

8. Demonstration of the Anti-Proliferative Effect of Christensenella Bacteria

The HT-29 cells were inoculated at a density of 10,000 cells/well in a total volume of 100 μl in a 96-well plate. After 24 hours of incubation, the culture medium was removed from the adherent cells and new medium supplemented with 10% Christensenella minuta stationary phase supernatant was added (DSMZ, C. minuta 1 and C. minuta 2 and in control GAM (bacterial culture medium)). Each condition was performed in 4 replicates. The cells were incubated for an additional 48 or 72 hours. Cell proliferation was determined by means of the CellTiter-Glo 2.0 assay kit (Promega). The measurements were carried out according to the manufacturer's instructions. Briefly, the plates were removed from the incubator and allowed to equilibrate at room temperature for 30 min and an equal volume of CellTiter-Glo 2.0 reagent was added directly into the wells (100 up. The plates were stirred at 300 rpm for 2 min using a rotary shaker, then incubated at room temperature for 10 min. The reaction mixture was then transferred to a white-walled 96-well plate and the luminescent signal was measured using a microplate reader (FLUOstar Omega, BMG Labtech).

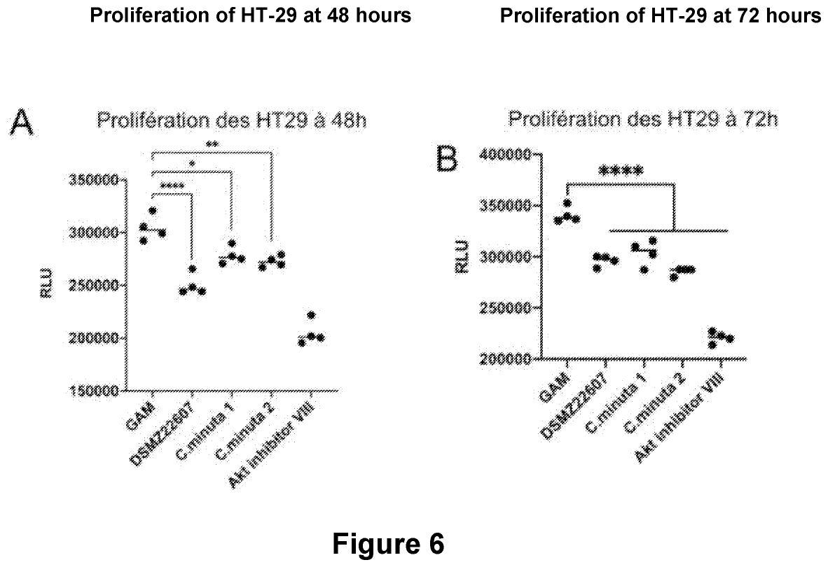

FIG. 6 shows the influence of strains of Christensenella minuta on the proliferation of tumor cells. The inventors observed the influence of strains of Christensenella minuta on the proliferation of tumor cells. We analyzed the effect of these supernatants by treating a human colon adenocarcinoma cell line, the HT-29 line. The effect of the supernatants of Christensenella minuta on the proliferation of HT-29 cells was evaluated after 48 hours and 72 hours. The supernatants of strains DSM22607 (DSMZ), C. minuta 1 and C. minuta 2 significantly reduce the proliferation of the cells treated for 48 hours and 72 hours, compared to the “GAM” control, corresponding to the cells treated with the bacterial culture medium (Statistical test=Dunnett where the control group is the GAM; ****GAM vs DSMZ p<0.0001, *GAM vs C. minuta 1 p=0.0140, **GAM vs C. minuta 2 p=0.0027 at 48 hours and ****p<0.0001 at 72 hours). Akt inhibitor VIII is used as a control for blocking cell proliferation.

This experiment makes it possible to conclude that the supernatants of the different strains of Christensenella minuta tested induce a decrease in the proliferation of the HT-29 tumor line, and therefore that these strains play a role in the inhibition of tumor development.

1. A bacterium of the genus Christensenella for use in the prevention and/or treatment of chronic inflammatory diseases and/or inflammatory gastrointestinal diseases and/or cancers in humans or animals.

2. The bacterium of the genus Christensenella for its use according to claim 1, in patients or animals exhibiting hyperproduction of interleukin 6 and/or interleukin 8 and/or underproduction of interleukin 10.

3. The bacterium of the genus Christensenella for its use according to claim 1, in the treatment of at least one disease selected from chronic inflammatory bowel diseases, chronic inflammatory diseases of the liver, chronic inflammatory diseases of the pancreas, polyarthritis, atopic dermatitis, neuroinflammatory diseases, chronic obstructive pulmonary disease and inflammatory bowel diseases.

4. The bacterium of the genus Christensenella for its use according to claim 1, in the treatment of at least one disease selected from Crohn's disease, ulcerative colitis, pouchitis, ulcerative colitis, celiac disease, autoimmune gastritis, hepatitis, nonalcoholic steatohepatitis, primary sclerosing cholangitis, pancreatitis, rheumatoid arthritis, psoriatic arthritis, psoriasis and eczema.

5. The bacterium of the genus Christensenella for its use according to claim 1, for use in the treatment of lymphomas, glioblastomas, myelomas, leukemias, colorectal cancers, breast cancers, cancers prostate, ovarian cancer, uterine cancer, pancreatic cancer, lung cancer, liver cancer, gallbladder cancer and kidney cancer.

6. The bacterium of the genus Christensenella for its use according to claim 1, characterized in that said bacterium is selected from Christensenella massiliensis, Christensenella timonensis, Christensenella intestinihominis, and Christensenella minuta.

7. A composition comprising at least one bacterium of the genus Christensenella according to claim 1 in a physiologically acceptable medium and/or a culture supernatant of at least one bacterial strain of the genus Christensenella for use in the prevention and/or the treatment of chronic inflammatory diseases and/or inflammatory gastrointestinal diseases and/or cancers.

8. The composition for its use according to claim 7, wherein said bacterium of the genus Christensenella and said culture supernatant may comprise or be derived from the same or different strains or species of Christensenella.

9. The composition for its use according to claim 7, said composition being administered to a human having a body mass index of less than 25.

10. The composition for its use according to claim 7, characterized in that said bacterium is selected from Christensenella massiliensis, Christensenella timonensis and Christensenella minuta and/or mixtures thereof.

11. The composition for its use according to claim 7, characterized in that at least 50% of the bacteria present are living bacteria (by number).

12. The composition for its use according to claim 7, characterized in that at least 90% of the bacteria present are living bacteria (by number).

13. The composition for its use according to claim 7, further comprising:
(i) at least one additional bacterium of the Verrucomicrobiaceae family; and/or
(ii) a culture supernatant of said additional bacterium.

14. The composition for its use according to claim 13, wherein the additional bacterial strain is a bacterium of the genus Akkermansia, more particularly of the species Akkermansia muciniphila.

15. The composition for its use according to claim 7, characterized in that it is provided in liquid form or in solid form.

16. The composition for its use according to claim 15, characterized in that the composition in solid form comprises said bacteria in lyophilized form.

17. The composition for its use according to claim 7, wherein the composition is administered orally, rectally, inhaled or topically.

18. The composition for its use according to claim 7, characterized in that it is in the form of powder, microencapsulated powder, gelatin capsule, capsule, tablet, lozenge, granules, emulsion, suspension, suppository, a beverage, a food, pharmaceutical or nutraceutical product, a food additive, dietary supplement or dairy product.

19. The composition for its use according to claim 7, characterized in that it is in a gastro-resistant form.

20. The composition for its use according to claim 7, characterized in that it comprises at least one probiotic and/or at least one prebiotic.

21. The composition for its use according to claim 7, characterized in that it also comprises:
at least one probiotic, and/or
at least one bacterium producing lactic acid and/or at least one other organism promoting the anaerobic conditions necessary for the survival of Christensenellaceae, and/or
at least one bacterium associated with the ecosystem of Christensenellaceae, and/or
at least one bacterium selected from bacteria of the genus Faecalibacterium, Akkermansia, Eubacterium and Oscillospira, and/or
at least one prebiotic, and/or
at least one polyphenol, and/or
at least one mineral and/or at least one vitamin, and/or at least one nutritional agent, and/or
at least one active pharmaceutical principle preferably selected from non-steroidal anti-inflammatory drugs, antibodies directed against pro-inflammatory targets (anti-TNFalpha, anti-1L-6, anti-1L-8), antirheumatic drugs, analgesics, antimicrobials, corticosteroids, anabolic steroids, antidiabetics, thyroid agents, antidiarrheals, cough suppressants, antiemetics, antiulcers, laxatives, anticoagulants, erythropoietin, immunoglobulins, immunosuppressants, growth hormones, hormonal drugs, estrogen receptor modulators, alkylating agents, antimetabolites, mitotic inhibitors, radiopharmaceuticals, anti-depressants, antipsychotics, anxiolytics, hypnotics, sympathomimetics, stimulants, donepezil, tacrine, asthma medications, beta agonists, inhaled steroids, leukotriene inhibitors, cromoglycates or cromoglycidic acids, epinephrine, alpha dornase, cytokines, cytokine antagonists, interleukin 10 agonists.

22. A composition comprising, in a physiologically acceptable medium:
(I) (a) at least one bacterial strain selected from the group consisting of Christensenella timonensis, Christensenella minuta and/or mixtures thereof; and/or
(b) at least one culture supernatant of at least one bacterial strain selected from
the group consisting of Christensenella timonensis, Christensenella minuta and/or mixtures thereof;
and
(II) (a) at least one additional bacterial strain of the genus Akkermansia, more particularly of the species Akkermansia muciniphila;
and/or
(b) at least one culture supernatant of at least one additional bacterial strain of the genus Akkermansia, more particularly of the species Akkermansia.


SEQUENCE LISTING (6 sequences)

SEQ ID NO: 1 - DNA, 1495 bp, Christensenella minuta
ggctcaggacgaacgctggcggcgtgcttaacacatgcaagtcgaacgaggttgccctttgtgaatccttcgggaggaactgtgggtataccgagtggcggacgggtgagtaacgcgtgagcaacctgccctgcaacgggggacaacagttggaaacgactgctaataccgcatgagaccacgaaaccgcatggttttgaggtaaaaggatttattcgatgcaggatgggctcgcgtcccattagatagttggtgaggtaacggcccaccaagtcaacgatgggtagccgacctgagagggtgatcggccacactggaactgagacacggtccagactcctacgggaggcagcagtggggaatattgggcaatgggggaaaccctgacccagcaacgccgcgtgagggaagaaggtcttcggattgtaaacctttgtcctatgggacgaaagaaatgacggtaccataggaggaagctccggctaactacgtgccagcagccgcggtaatacgtagggagcaagcgttgtccggaattactgggcgtaaagggtgcgtaggtggtcatgtaagtcagatgtgaaagaccggggcttaaccccggggttgcatttgaaactgtgtgacttgagtacaggagagggaagtggaattcctagtgtagcggtgaaatgcgtagatattaggaggaacaccagtggcgaaggcgactttctggactgtaactgacactgaggcacgaaagcgtggggagcaaacaggattagataccctggtagtccacgccgtaaacgatggatactaggtgtggggcccgatagggttccgtgccgaagcaaacgcattaagtatcccgcctggggagtacgatcgcaaggttgaaactcaaaggaattgacgggggcccgcacaagcagcggagcatgtggtttaattcgaagcaacgcgaagaaccttaccaaggcttgacatcctctgacgactgtagagatacagtttcccttcggggcagagagacaggtggtgcatggttgtcgtcagctcgtgtcgtgagatgttgggttaagtcccgcaacgagcgcaacccttattgctagttgccagcgcgtaaaggcgggaactctagtgagactgccggggacaactcggaggaaggtggggacgacgtcaaatcatcatgccccttatgtcttgggctacacacgtgctacaatggccggtacaaagggcagcgaacccgtaaggggaagcgaatctcaaaaagccggtcccagttcggattgtgggctgcaacccgcccacatgaagtcggagttgctagtaatcgcgaatcagcatgtcgcggtgaatgcgttcccgggccttgtacacaccgcccgtcacaccacggaagttgggagcacccgaagccagtggcttaaccgtaaggagagagctgtcgaaggtgagatcaatgactggggtgaagtcgtaacaaggtagccgtatcggaagg

SEQ ID NO: 2 - DNA, 292 bp, Christensenella minuta
gtgccagcagccgcggtaatacgtagggagcaagcgttgtccggaattactgggcgtaaagggtgcgtaggtggtcatgtaagtcagatgtgaaagaccggggcttaaccccggggttgcatttgaaactgtgtgacttgagtacaggagagggaagtggaattcctagtgtagcggtgaaatgcgtagatattaggaggaacaccagtggcgaaggcgactttctggactgtaactgacactgaggcacgaaagcgtggggagcaaacaggattagataccctggtagtcc

SEQ ID NO: 3 - DNA, 1357 bp, Christensenella minuta
  misc_feature 550..554: /note=“n is a, c, g, or t”
  misc_feature 568..569: /note=“n is a, c, g, or t”
  misc_feature 605..605: /note=“n is a, c, g, or t”
  misc_feature 612..612: /note=“n is a, c, g, or t”
  misc_feature 617..619: /note=“n is a, c, g, or t”
ggacgaacgctggcggcgtgcttaacacatgcaagtcgaacgaggtgcagcgagcggaccccttcggggagaagcatgctgtatcctagtggcggacgggtgagtaacgcgtgagcaacctacccttgaatgggggacaacagctggaaacggctgctaataccgcataagaccacgtcatcgcatggtgaagaggtaaaaggattaattcgatcaaggatgggctcgcgtcccattagatagttggtgagatagcagctcaccaagccgacgatgggtagccgacctgagagggtgaacggccacactggaactgagacacggtccagactcctacgggaggcagcagtggggaatattgggcaatgggggaaaccctgacccagcaacgccgcgtgaaggaagaaggccttcgggttgtaaacttttgtcctatgggacgaagaagtgacggtaccataggaggaagctccggctaactacgtgccagcagccgcggtaatacgtagggagcgagcgttgtccggaattactgggcgtaaagggtgcnnnnncggagatgtaagtnnggtgtgaaagcccggggctcaaccccgggattgcanttgaaantacannncttgagtacaggagaggcaagtggaattcctagtgtagcggtgaaatgcgtagatattaggaggaacaccagtggcgaaggcgacttgctggactgtaactgacgctgaggcacgaaagcgtggggagcaaacaggattagataccctggtagtccacgccgtaaacgatggatactaggtgtaggggtcgataggcctctgtgccgaagcaaacgcattaagtatcccgcctggggagtacgatcgcaaggttgaaactcaaaggaattgacgggggcccgcacaagcagcggagcatgtggtttaattcgaagcaacgcgaagaaccttaccaaggcttgacatcctctgacggctatagagatatagcttcccttcggggcagagagacaggtggtgcatggttgtcgtcagctcgtgtcgtgagatgttgggttaagtcccgcaacgagcgcaacccttattgctagttgccagcacgtaaaggtgggaactctagtgagaccgccggggacaactcggaggaaggtggggacgacgtcaaatcatcatgccccttatgtcttgggctacacacgtgctacaatggccggtacaaagggtagcgaagtcgtaagatgaagcgaatctcaaaaagccggtcccagttcggattgagggctgcaacccgccctcatgaagtcggagttgctagtaatcgcgaatcagcatgtcgcggtgaatgcgttcccgggcct

SEQ ID NO: 4 - DNA, 1402 bp, Christensenella minuta
ttcgcccttagagtttgatcctggctcaggacgaacgctggcggcgtgcttaacacatgcaagtcgaacgaagttgctctttgggaagccctcgggtggaactgtgagtatacttagtggcggacgggtgagtaacgcgtgagcaatctgccctgcaatgggggacaacagttggaaacgactgctaatgccgcataagaccacgaaaccgcatggtttagaggtaaaaggattcattcgatgcaggatgagctcgcgtcccattagatagttggtgaggtaacggcccaccaagtcaacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttggggaagataatgacggtgcccaacaaggaagtgacggctaactacgtgccagcagccgcggtaatacgtagggagcaagcgttgtccggaattactgggcgtaaagggtgcgtaggtggccatgtaagtcaggtgtgaaagaccggggctcaaccccggggttgcacttgaaactgtgtggcttgagtacaggagagggaagtggaattcctagtgtagcggtgaaatgcgtagatattaggaggaacaccagtggcgaaggcgactttctggactgtaactgacactgaggcacgaaagcgtggggagcaaacaggattagataccctggtagtccacgccgtaaacgatggatactaggtgtgggggggcgatagtcctccgtgccgaagctaacgcattaagtatcccgcctggggagtacgatcgcaaggttgaaactcaaaggaattgacgggggcccgcacaagcagcggagcatgtggtttaattcgaagcaacgcgaagaaccttaccaaggcttgacatcctctgacgcatatagagatatatgttcccttcggggcagagagacaggtggtgcatggttgtcgtcagctcgtgtcgtgagatgttgggttaagtcccgcaacgagcgcaacccttgttgctagttgccagcacgtaaaggtgggaactctagtgagactgccggggacaactcggaggaaggtggggacgacgtcaaatcatcatgccccttatgtcttgggctacacacgtgctacaatggccggtacaaagggcagcgacctcgtaagagtaagcgaatctcaaaaagccggccccagttcggattgtgggctgcaacccgcccacatgaagtcggagttgctagtaatcgcgaatcagcatgtcgcggtgaatgcgttcccgggccttgcacacaccgcccgtca

SEQ ID NO: 5 - DNA, 1497 bp, Christensenella minuta
agagtttgatcctggctcaggacgaacgctggcggcgtgcttaacacatgcaagtcgaacgaagttgctctttgggaagccctcgggtggaactgtgagtatacttagtggcggacgggtgagtaacgcgtgagcaatctgccctgcaatgggggacaacagttggaaacgactgctaataccgcataagaccacgaaaccgcatggtttagaggtaaaaggattcattcgatgcaggatgagctcgcgtcccattagatagttggtgaggtaacggcccaccaagtcaacgatgggtagccgacctgagagggtgatcggccacattggaactgagaaacggtccaaactcctacgggaggcagcagtggggaatattgggcaatgggggaaaccctgacccagcaacgccgcgtgaaggaagaaggccttcgggttgtaaacttttgtcctatgggacgaaaaaaatgacggtaccataggaggaagctccggctaactacgtgccagcagccgcggtaatacgtagggagcaagcgttgtccggaattactgggcgtaaagggtgcgtaggtggccatgtaagtcaggtgtgaaagaccggggctcaaccccggggttgcacttgaaactgtgtggcttgagtacaggagagggaagtggaattcctagtgtagcggtgaaatgcgtagatattaggaggaacaccagtggcgaaggcgactttctggactgtaactgacactgaggcacgaaagcgtggggagcaaacaggattagataccctggtagtccacgccgtaaacgatggatactaggtgtggggggcgatagtcctccgtgccgaagctaacgcattaagtatcccgcctggggagtacgatcgcaaggttgaaactcaaaggaattgacgggggcccgcacaagcagcggagcatgtggtttaattcgaagcaacgcgaagaaccttaccaaggcttgacatcctctgacgcatatagagatatatgttcccttcggggcagagagacaggtggtgcatggttgtcgtcagctcgtgtcgtgagatgttgggttaagtcccgcaacgagcgcaacccttgttgctagttgccagcacgtaaaggtgggaactctagtgagactgccggggacaactcggaggaaggtggggacgacgtcaaatcatcatgccccttatgtcttgggctacacacgtgctacaatggccggtacaaagggcagcgacctcgtaagagtaagcgaatctcaaaaagccggccccagttcggattgtgggctgcaacccgcccacatgaagtcggagttgctagtaatcgcgaatcagcatgtcgcggtgaatgcgttcccgggccttgtacacaccgcccgtcacaccacggaagttgggagcacccgaagccagtggcttaaccgtaaggagagagctgtcgaaggtgagatcaatgactggggtgaagtcgtaacaaggtaacc

SEQ ID NO: 6 - DNA, 1495 bp, Christensenella minuta
agagtttgatcctggctcaggacgaacgctggcggcgtgcttaacacatgcaagtcgaacgaagatgctctttgtgaagccttcgggtggaactgtgagtagactgagtggcggacgggtgagtaacgcgtgagcaacctgccctgcaacgggggacaacagctggaaacggctgctaataccgcataagaccacggaatcgcatggttctgaggtaaaaggatttattcgatgcaggatgggctcgcgtcccattagatagttggtgaggtaacggctcaccaagtcaacgatgggtagccgacctgagagggtgatcggccacactggaactgagacacggtccagactcctacgggaggcagcagtggggaatattgggcaakgggcgagcctgaccagcacgccgcgtgaaggaagaaggccttcgggttgtaaacttttgtcctatgggaagaagaagtgacggtaccataggaggaagctccggctaactacgtgccagcagccgcggtaatacgtagggagcaagcgttgtccggaattactgggcgtaaagggtgcgtaggcggtttgacaagtcagatgtgaaagcctggggcttaactccaggattgcatttgaaactgttaaacttgagtgcaggagaggcaagtggaattcctagtgtagcggtgaaatgcgtagatattaggaggaacaccagtggcgaaggcgacttgctggactgtaactgacgctgaagcacgaaagcgtggggagcaaacaggattagataccctggtagtccacgctgtaaacgatggatactaggtgtagggggcgatagtcttctgtgccgcagctaacgcattaagtatcccgcctggggagtacgatcgcaaggttgaaactcaaaggaattgacgggggcccgcacaagcagcggagcatgtggtttaattcgaagcaacgcgaagaaccttaccaaggcttgacatcctctgacgcatgtagagatacatgttcccttcggggcagagagacaggtggtgcatggttgtcgtcagctcgtgtcgtgagatgttgggttaagtcccgcaacgagcgcaacccttattgctagttgccagcacgtaaaggtgggaactctagtgagaccgccggggacaactcggaggaaggtggggacgacgtcaaatcatcatgccccttatgtcttgggctacacacgtgctacaatggccggtacaaaggacagcgaacccgcaaggggaagcgaatctcaaaaagccggtcccagttcggattgagggctgcaacccgccctcatgaagtcggagttgctagtaatcgcgaatcagcatgtcgcggtgaatgcgttcccgggccttgtacacaccgcccgtcacaccacggaagttgggagcgcccgaagccagtggcttaaccgtaaggagagagctgtcgaaggtgagatcaatgactggggtgaagtcgtaacaaggtaaccgta